(19) United States
(12) Patent Application Publication
(10) Pub. No.: US 2008/0241127 A1
(43) Pub. Date: Oct. 2, 2008

(54) Methods of Making Passive Immunotherapies

(75) Inventors: Henry Blair, Boston, MA (US); Clive R. Wood, Boston, MA (US); Robert C. Ladner, Ijamsville, MD (US)

Correspondence Address: LOWRIE, LANDO & ANASTASI, LLP, ONE MAIN STREET, SUITE 1100, CAMBRIDGE, MA, 02142, US

(73) Assignee: DYAX CORP.

(21) Appl. No.: 11/955,595

(22) Filed: Dec. 13, 2007

Related U.S. Application Data
(60) Provisional application No. 60/877,840, filed on Dec. 29, 2006.

(51) Int. Cl. A61K 39/395; C40B 30/04

(52) U.S. Cl. 424/130.1; 506/9

(57) ABSTRACT

Methods for isolating antibodies useful as passive immunotherapies are described.

This application claims priority to U.S. Application Ser. No. 60/877,840, filed on Dec. 29, 2006, the contents of which is incorporated by reference in its entirety.

### BACKGROUND

Vaccines for inducing immunity against a wide variety of disease-causing agents and toxins are available for administration to humans. However, a significant period of time (weeks to months) is required for the immune system of the vaccinated subject to generate a substantial immune response against the vaccine (and thus against the toxin or disease-causing agent).

Passive immunotherapy (administration of antibodies or fractionated serum from immune donors) has been used to provide protection against diseases or toxins when either it is infeasible to wait for the subject to develop an immune response against the vaccine or when the subject has a compromised immune system and is unlikely to develop a sufficiently robust response to the vaccine.

### SUMMARY OF THE INVENTION

Passive immunotherapies are derived from the immune serum of a subject (human or non-human animal) that has been vaccinated against or exposed to the disease-causing agent or toxin. The serum is typically fractionated and treated to reduce the chances of transmission of infectious agents. There are a number of drawbacks to this process, including concerns regarding immunogenicity of the immunotherapeutic (especially when the passive immunotherapeutic is derived from a different species than the subject receiving the immunotherapy), concerns regarding transmission of infectious agents (especially when the immunotherapy is derived from human serum), and reluctance of immune humans to repeatedly donate serum.

The inventors have created new methods for producing passive immunotherapies which avoid the drawbacks of traditional passive immunotherapeutic production by selecting the antibodies which make up the passive immunotherapy by selection of antibodies in vitro using antibody display technology, thereby eliminating the use of immune serum from animals or other immune donors. The use of antibody display technology allows the production of species-matched antibodies (e.g., human antibodies for administration to a human) and eliminates the possibility of transmission of infectious agents from donor immune serum to the subject receiving the passive immunotherapy. The invention provides an additional advantage of decreased risk to the workers producing the passive immunotherapeutic because the handling of human-derived serum products is greatly reduced or eliminated.

Accordingly, disclosed herein are new methods for identifying antibodies for use in conferring passive immunity to a toxin or disease-causing agent and for making immunotherapeutic compositions. A vaccine (or the active ingredient thereof) is used to select one or more antibodies from a display library of antibodies (e.g., a plurality of different antibodies displayed on the surface replicable genetic packages, e.g., a library of bacteriophage displaying Fabs). The selected antibodies may be further screened for affinity and for additional activities, such as enzyme inhibition (e.g., when a particular enzymatic activity is important to infectiousness, virulence, or toxicity of the disease-causing agent or toxin). The antibodies thus identified are used to produce the passive immunotherapeutic.

In one aspect, the invention provides a method for identifying one or more antibodies for use in an passive immuntherapy, by selecting antibodies from an antibody display library which bind to a target vaccine. The selection is carried out by contacting the target vaccine with members of the antibody display library and identifying the members of the antibody display library which bind the target vaccine. The target vaccine may be a cellular vaccine (e.g., using whole viruses or cells) or an acellular vaccine (e.g., using cell-free cellular extracts, cell-free purified or fractionated cellular components, such as protein(s) or polysaccharides (e.g., cell wall components), or conjugates of purified or fractionated cellular components to a carrier moiety, such as a carrier protein).

In some embodiments, the target vaccine is a cellular vaccine that is a live vaccine (e.g., incorporating microorganisms (e.g., bacteria or viruses) which are viable and capable of reproduction). Exemplary live vaccines include anthrax (Bacillus anthracis) vaccines (e.g., Stern-strain vaccines, such as those for veterinary use), smallpox vaccines (e.g., live vaccinia virus vaccines), rotavirus vaccines (e.g., live multivalent vaccines containing rhesus monkey and/or rhesus/human reassortment rotavirus, such as ROTASHIELD® and ROTATEQ®).

In some embodiments, the target vaccine is a cellular vaccine that is an attenuated vaccines (e.g., incorporating microorganisms which are viable or otherwise able to reproduce but selected or otherwise treated to reduce virulence, e.g., the Sabin oral polio vaccine). Exemplary attenuated vaccines include Sabin oral polio vaccine (OPV), attenuated influenza A/B vaccines (e.g., FLUMIST®), measles vaccine (e.g., ATTENUVAX®), mumps vaccine (e.g., vaccines containing Jeryl Lynn strain attenuated mumps virus), rubella or “german measles” vaccine (e.g., ERVAX®, ERVEVAX®, MERUVAX®, or GUNEVAX®), and chickenpox/shingles vaccine (e.g., attenuated varicella zoster virus, such as ZOSTAVAX®, VARVAX®, or VARILRIX®).

In some embodiments, the target vaccine is a cellular vaccine that is a killed vaccines (e.g., incorporating microorganisms or infectious agents which have been killed or inactivated, e.g., the Salk polio vaccine). Exemplary killed vaccines include inactivated hepatitis A whole virus vaccines (e.g., HAVRIX® and VAQTA®), the Salk polio vaccine, injected influenza A/B vaccines, and killed rabies virus vaccines (e.g., RABIVAC®, RABAVERT®).

In some embodiments, the target vaccine is an acellular vaccine (e.g., incorporating cell-free components or cellular extracts). In some embodiments, the acellular vaccine includes a toxoid (an inactivated toxin), a cell wall component, and/or a proteinaceous component of the microorganism or infectious agent targeted by the vaccine. In some embodiments, the active component of the target vaccine (e.g., the toxoid, cell wall component, and/or proteinaceous antigen) is adsorbed (e.g., to alum) or is conjugated to a carrier moiety, such as diptheria toxoid). Exemplary acellular vaccines include acellular anthrax vaccine (e.g., BIOTHRAX™, an acellular extract of Bacillus anthracis conditioned growth medium), hepatitis B surface antigen vaccines (e.g., ENGERIX-B® or RECOMBIVAX HB®), diptheria toxoid vaccines (e.g., formaldehyde-inactivated diptheria toxin vaccines, which may be adsorbed, such as to alum), Haemophilus influenzae type B capsular polysaccharide conjugate vaccines (e.g., capsular polysaccharide/diptheria toxin CRM₁₉₇ conjugate (e.g., HibTITER®), capsular polysaccharide/tetanus toxoid conjugate (e.g. ACTHIB®), or capsular polysaccharide/Neisseria menigitidis OmpC conjugate vaccines (e.g., PEDVAXHIB®)), Streptococcus pneumoniae capsular polysaccharide vaccines (e.g., multi-valent capsular polysaccharide vaccines (e.g., PNEUMOVAX® 23) or capsular polysaccharide/diptheria toxin CRM₁₉₇ conjugate vaccines (e.g., PREVNAR®)), Neisseria meningitidis multivalent capsular extract vaccines (e.g., A, C, Y, W-135 capsular polysaccharide extract vaccines (e.g., MENOMUNE®) or A, C, Y, W-135 capsular polysaccharide extract/diptheria toxoid conjugate vaccines (e.g., MENACTRA™)), pertussis vaccines (e.g., vaccines including pertussis toxoid, filamentous hemagglutinin and/or pertactin), tetanus vaccines (e.g., Clostricium tetani tetanus toxoid vaccines, which may be adsorbed, such as to alum), typhus vaccines (e.g., Salmonella typhi capsular polysaccharide vaccines (e.g., TYPHIM VI® or TYPHERIX®), Lyme's disease vaccines (e.g., Borrelia burgdorferi lipoprotein OspA vaccines (e.g., LYMERIX™)), human papillomavirus (HPV) vaccines (e.g., vaccines containing viruslike particles of the major capsid protein (L1) of HPV (e.g., viruslike particles of L1 from HPV 6, 11, 16, and 18, e.g., GARDASIL®), ricin toxin or toxoid (e.g., mutant ricin toxin vaccines, such as those containing ricin V76M/Y80A (RIVAX™) or ricin toxoid), and Clostridium botulinum (e.g., botulinum) toxin or toxoid (e.g., vaccines containing the botulinum toxin heavy chain).

In one embodiment, the step of identifying members of the antibody display library which bind the target vaccine includes eluting bound display library members from the target vaccine by bulk elution (e.g., by altering the chemical properties of the reaction containing the antibody/target vaccine complexes, such as by altering pH or salt conditions), which elutes (disrupts the target vaccine/display library member complexes) essentially all of the antibody display library members bound to the target vaccine.

In another embodiment, the step of identifying the members of the antibody display library which bind the target vaccine includes eluting the bound display library members from the target vaccine by competition, using a competition reagent containing molecules which bind to the target vaccine. The competition reagent may be known antibodies to the active agent(s) in the target vaccine (e.g., the molecules, extracts, microorganisms or infectious agents in the target vaccine which act as immunogens), or it may be immune serum derived from one or more individuals who have been exposed to the molecule, microorganism, or infectious agent targeted by the target vaccine (e.g., a subject who has been immunized with the target vaccine or a subject who has been exposed to or infected by molecule, microorganism, or infectious agent targeted by the target vaccine).

In one embodiment, the step of identifying members of the antibody display library which bind the target vaccine also includes obtaining the nucleotide sequence(s) of the nucleic acid(s) which encodes the antibody displayed on the surface of antibody display library members being identified.

In some embodiments, additional steps of characterization are carried out, including testing the affinity and/or specificity of the selected antibody for the target vaccine.

In some embodiments, the target vaccine is a univalent (e.g., containing only a single strain of cellular microorganism or infectious agent or single immunogen, such as a toxin or cell wall extract). In other embodiments, the target vaccine is multivalent (e.g., polio vaccines, which are trivalent or containing three strains of polio virus).

In another aspect, the invention provides passive immunotherapeutic compositions which incorporate one or more antibodies identified by the methods disclosed herein.

In another aspect, the invention provides kits which include a passive immunotherapeutic composition incorporating one or more antibodies identified by the methods disclosed herein.

In a further aspect, the invention provides methods of prophylaxis or treatment of a disorder targeted by a target vaccine. A passive immunotherapeutic incorporating antibodies identified by the methods disclosed herein can be administered to a subject at risk of, exposed to, or having the disease or disorder targeted by the target vaccine. In some embodiments, the passive immunotherapeutic is administered to an immunocompromised individual.

In some embodiments, a passive immunotherapeutic incorporating antibodies identified by the methods disclosed herein is administered to a subject at risk of, exposed to, or having a disease such as anthrax (Bacillus anthracis infection), hepatitis A, hepatitis B, polio, smallpox, bacterial meningitis (Streptococcus pneumoniae, Neisseria meningitidis, or Haemophilus influenzae infection), diptheria (Corynebacterium diphtheriae infection), influenza, measles, mumps, pertussis (Bordetella pertussis infection), pneumococcal pneumonia (Streptococcus pneumoniae infection), ricin toxin intoxication, botulisim, tetanus (Clostricium tetani infection), rotaviral diarrhea (rotavirus infection), rubella, typhus, rabies, human papillomavirus (HPV) infection, cervical cancer associated with HPV infection, chickenpox, or shingles.

### DETAILED DESCRIPTION

The term “antibody” refers to a protein that includes at least one immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab and sFab fragments, F(ab′)₂, Fd fragments, Fv fragments, scFv, and domain antibodies (dAb) fragments (de Wildt et al., Eur J. Immunol. 1996; 26(3):629-39)) as well as complete antibodies. An antibody can have the structural features of IgA, IgG, IgE, IgD, IgM (as well as subtypes thereof). Antibodies may be from any source, but primate (human and non-human primate) and primatized are preferred

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, see also www.hgmp.mrc.ac.uk). Kabat definitions are used herein. Each VH and VL is typically composed of three CDRs and four FRs, arranged from amino-terminus to carboxy-terminus in the following order: FR1, CDR1, FR2, CDR2, FR3, CDR3, FR4.

As used herein, an “immunoglobulin variable domain sequence” refers to an amino acid sequence which can form the structure of an immunoglobulin variable domain such that one or more CDR regions are positioned in a conformation suitable for an antigen binding site. For example, the sequence may include all or part of the amino acid sequence of a naturally-occurring variable domain. For example, the sequence may omit one, two or more N- or C-terminal amino acids, internal amino acids, may include one or more insertions or additional terminal amino acids, or may include other alterations. In one embodiment, a polypeptide that includes immunoglobulin variable domain sequence can associate with another immunoglobulin variable domain sequence to form an antigen binding site.

The VH or VL chain of the antibody can further include all or part of a heavy or light chain constant region, to thereby form a heavy or light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. In IgGs, the heavy chain constant region includes three immunoglobulin domains, CH1, CH2 and CH3. The light chain constant region includes a CL domain. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system. The light chains of the immunoglobulin may be of types kappa or lambda. In one embodiment, the antibody is glycosylated. An antibody can be functional for antibody-dependent cytotoxicity and/or complement-mediated cytotoxicity. The Fc region can be human.

One or more regions of an antibody can be human or effectively human. For example, one or more of the variable regions can be human or effectively human. For example, one or more of the CDRs can be human, e.g., HC CDR1, HC CDR2, HC CDR3, LC CDR1, LC CDR2, and LC CDR3. Each of the light chain CDRs can be human. HC CDR3 can be human. One or more of the framework regions can be human, e.g., FR1, FR2, FR3, and FR4 of the HC or LC.

In one embodiment, all the framework regions are human, e.g., derived from a human somatic cell, e.g., a hematopoietic cell that produces immunoglobulins or a non-hematopoietic cell. In one embodiment, the human sequences are germline sequences, e.g., encoded by a germline nucleic acid. In one embodiment, the framework (FR) residues of a selected Fab can be convertered to the amino-acid type of the corresponding residue in the most similar primate germline gene, especially the human germline gene. One or more of the constant regions can be human or effectively human. For example, at least 70, 75, 80, 85, 90, 92, 95, 98, or 100% of an immunoglobulin variable domain, the constant region, the constant domains (CH1, CH2, CH3, CL1), or the entire antibody can be human or effectively human.

All or part of an antibody can be encoded by an immunoglobulin gene or a segment thereof. Exemplary human immunoglobulin genes include the kappa, lambda, alpha (IgA1 and IgA2), gamma (IgG1, IgG2, IgG3, IgG4), delta, epsilon and mu constant region genes, as well as the many immunoglobulin variable region genes. Full-length immunoglobulin “light chains” (about 25 KDa or about 214 amino acids) are encoded by a variable region gene at the NH2-terminus (about 110 amino acids) and a kappa or lambda constant region gene at the COOH— terminus. Full-length immunoglobulin “heavy chains” (about 50 KDa or about 446 amino acids), are similarly encoded by a variable region gene (about 116 amino acids) and one of the other aforementioned constant region genes, e.g., gamma (encoding about 330 amino acids). The length of human HC varies considerably because HC CDR3 varies from about 3 amino-acid residues to over 35 amino-acid residues.

The term “antigen-binding fragment” of a full length antibody refers to one or more fragments of a full-length antibody that retain the ability to specifically bind to a target of interest. Examples of binding fragments encompassed within the term “antigen-binding fragment” of a full length antibody include (i) a Fab fragment, a monovalent fragment consisting of the VL, VH, CL and CH1 domains; (ii) a F(ab′)₂ fragment, a bivalent fragment including two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR) that retains functionality. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv). See e.g., U.S. Pat. Nos. 5,260,203, 4,946,778, and 4,881,175; Bird et al. (1988) Science 242:423-426; and Huston et al. (1988) Proc. Natl. Acad. Sci. USA 85:5879-5883.

Antibody fragments can be obtained using any appropriate technique including conventional techniques known to those with skill in the art. The term “monospecific antibody” refers to an antibody that displays a single binding specificity and affinity for a particular target, e.g., epitope. This term includes a “monoclonal antibody” or “monoclonal antibody composition,” which as used herein refer to a preparation of antibodies or fragments thereof of single molecular composition, irrespective of how the antibody was generated.

An “effectively human” immunoglobulin variable region is an immunoglobulin variable region that includes a sufficient number of human framework amino acid positions such that the immunoglobulin variable region does not elicit an immunogenic response in a normal human. An “effectively human” antibody is an antibody that includes a sufficient number of human amino acid positions such that the antibody does not elicit an immunogenic response in a normal human.

A “humanized” immunoglobulin variable region is an immunoglobulin variable region that is modified to include a sufficient number of human framework amino acid positions such that the immunoglobulin variable region does not elicit an immunogenic response in a normal human. Descriptions of “humanized” immunoglobulins include, for example, U.S. Pat. No. 6,407,213 and U.S. Pat. No. 5,693,762.

As used herein, “binding affinity” refers to the apparent association constant or Kₐ. The Kₐ is the reciprocal of the dissociation constant (K_{d}). A binding protein may, for example, have a binding affinity of at least 10⁵, 10⁶, 10⁷, 10⁸, 10⁹, 10¹⁰ and 10¹¹ M⁻¹ for a particular target molecule, e.g., MMP-14, MMP-16, or MMP-24. Higher affinity binding of a binding protein to a first target relative to a second target can be indicated by a higher Kₐ (or a smaller numerical value K_{d}) for binding the first target than the Kₐ (or numerical value K_{d}) for binding the second target. In such cases, the binding protein has specificity for the first target (e.g., a protein in a first conformation or mimic thereof) relative to the second target (e.g., the same protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 91, 100, 500, 1000, or 10⁵ fold.

Binding affinity can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in TRIS-buffer (50 mM TRIS, 150 mM NaCl, 5 mM CaCl₂ at pH7.5). These techniques can be used to measure the concentration of bound and free binding protein as a function of binding protein (or target) concentration. The concentration of bound binding protein ([Bound]) is related to the concentration of free binding protein ([Free]) and the concentration of binding sites for the binding protein on the target where (N) is the number of binding sites per target molecule by the following equation:

[Bound]=N·[Free]/((1/Ka)+[Free]).

It is not always necessary to make an exact determination of Kₐ, though, since sometimes it is sufficient to obtain a quantitative measurement of affinity, e.g., determined using a method such as ELISA or FACS analysis, is proportional to Kₐ, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

An “isolated composition” refers to a composition that is removed from at least 90% of at least one component of a natural sample from which the isolated composition can be obtained. Compositions produced artificially or naturally can be “compositions of at least” a certain degree of purity if the species or population of species of interests is at least 5, 10, 25, 50, 75, 80, 90, 92, 95, 98, or 99% pure on a weight-weight basis.

An “epitope” refers to the site on a target compound that is bound by a binding protein (e.g., an antibody such as a Fab or full length antibody). In the case where the target compound is a protein, the site can be entirely composed of amino acid components, entirely composed of chemical modifications of amino acids of the protein (e.g., glycosyl moieties), or composed of combinations thereof. Overlapping epitopes include at least one common amino acid residue, glycosyl group, phosphate group, sulfate group, or other molecular feature.

Statistical significance can be determined by any art known method. Exemplary statistical tests include: the Students T-test, Mann Whitney U non-parametric test, and Wilcoxon non-parametric statistical test. Some statistically significant relationships have a P value of less than 0.05 or 0.02. Particular binding proteins may show a difference, e.g., in specificity or binding, that are statistically significant (e.g., P value <0.05 or 0.02). The terms “induce”, “inhibit”, “potentiate”, “elevate”, “increase”, “decrease” or the like, e.g., which denote distinguishable qualitative or quantitative differences between two states, and may refer to a difference, e.g., a statistically significant difference, between the two states.

As used herein, the terms “treatment” or “treating,” as used herein with reference to a disease or disorder, means eliminating, reducing, improving, or stabilizing at least one symptom of a particular disease or disorder. The terms “prophylaxis” and “preventing,” as used herein with reference to a disease or disorder, means reducing the prevelance of the disease or disorder in a particular population, reducing the risk of a particular subject contracting the disease or disorder, or reducing the severity of the disease or disorder in a subject (when the prophylactic treatment is initiated prior to the subject developing a symptom of the disease or disorder).

Display Libraries

A display library is a collection of entities; each entity includes an accessible polypeptide component and a recoverable component that encodes or identifies the polypeptide component (e.g., a DNA sequence encoding the polypeptide component), and in some cases the entity includes more than one polypeptide component, for example, the two polypeptide chains of a Fab (along with the recoverable element that encodes or identifies the polypeptide components). The polypeptide component is varied so that different amino acid sequences are represented.

Display libraries for use in the instant invention display antibodies, preferably in a two chain format (e.g., Fabs), although display libraries for use in the methods of the invention may display other forms of antibodies (e.g., single domain, or camelid, antibodies, or single chain, or scFv, antibodies). The displayed antibodies may include one or more constant regions in addition to the variable region (in the case of antibodies displayed in a two chain format, e.g., Fabs, each chain may include one or more constant domains, and in one embodiment, each chain includes one constant domain.

The displayed antibodies may be of any species, but it is generally preferred that the displayed antibodies be derived (in whole or in part) from the species for which the passive immunotherapeutic is intended (e.g., the displayed antibodies are “species-matched” to the intended recipient). For example, when the passive immunotherapeutic is intended for administration to humans, the display library preferably display human antibodies or antibodies that are effectively human (e.g., antibodies that have some, a majority, or all of the framework regions derived from human antibodies). Libraries of humanized or effectively human antibodies may include CDR sequences captured from other species, such as primates (e.g., chimapanzee, bonobo, macaque), rodents (e.g., rats, mice), ovines, bovines, equines, camelids, or any other species of interest. Alternately, the displayed antibodies may be non-species-matched (e.g., a mouse antibody library for selection of antibodies for administration to humans), and the selected antibodies are later ‘humanized’ by replacing sequences of the Fv variable region that are not directly involved in antigen binding with equivalent sequences from human Fv variable regions (for methods of humanization, see, e.g., Morrison, S. L., 1985, Science 229:1202-1207, by Oi et al., 1986, BioTechniques 4:214, and by Queen et al. U.S. Pat. No. 5,585,089, U.S. Pat. No. 5,693,761 and U.S. Pat. No. 5,693,762).

The nucleic acid sequences encoding the displayed antibodies may be captured (e.g., isolated, amplified, or otherwise derived from a sample of cells (e.g., peripheral blood leukocytes (PBLs), such as by PCR amplification of the VH and VL sequences of the PBLs in the sample), partially captured (e.g., incorporate CDR sequences captured from PBLs combined with framework(s) from an alternate source, such as from the sequence of a known germline antibody structure, e.g., DP47), or wholly synthetic (e.g., synthetic CDRs combined with framework sequences from an alternate source, such as from the sequence of a known germline antibody, e.g., DP47), or a combination thereof (e.g., captured VL sequences combined with synthetic VH or semi-synthetic VH (see, e.g., Hoet et al. (2005) Nat. Biotechnol. 23(3)344-8, which combines captured VL genes with VH made with synthetic CDR1 and CDR2 sequences, framework regions from germline antibody DP47, and CDR3 sequences captured from autoimmune patients).

Antibody libraries can be constructed by a number of processes (see, e.g., de Haard et al., 1999, J. Biol. Chem. 274:18218-30; Hoogenboom et al., 1998, Immunotechnology 4:1-20; Hoogenboom et al., 2000, Immunol. Today 21:371-378, and Hoet et al. (2005) Nat. Biotechnol. 23(3)344-8. Further, elements of each process can be combined with those of other processes. The processes can be used such that variation is introduced into a single immunoglobulin domain (e.g., VH or VL) or into multiple immunoglobulin domains (e.g., VH and VL). The variation can be introduced into an immunoglobulin variable domain, e.g., in the region of one or more of CDR1, CDR2, CDR3, FR1, FR2, FR3, and FR4, referring to such regions of either and both of heavy and light chain variable domains. The variation(s) may be introduced into all three CDRs of a given variable domain, or into CDR1 and CDR2, e.g., of a heavy chain variable domain. Any combination is feasible. In one process, antibody libraries are constructed by inserting diverse oligonucleotides that encode CDRs into the corresponding regions of the nucleic acid. The oligonucleotides can be synthesized using monomeric nucleotides or trinucleotides. For example, Knappik et al., 2000, J. Mol. Biol. 296:57-86 describe a method for constructing CDR encoding oligonucleotides using trinucleotide synthesis and a template with engineered restriction sites for accepting the oligonucleotides.

When captured VH or VL (or portions thereof, such as captured CDR sequences) are used to construct libraries, the nucleic acids encoding the captured sequences can be harvested from naïve germline immunoglobin genes, or from non-naïve cells, which may be obtained from a subject that has or has not been exposed to the target vaccine (e.g., is naïve or non-naïve with respect to the target vaccine). In one embodiment, the immunoglobin gene sequences are harvested from a non-human animal that includes a human immunoglobin locus.

A variety of formats can be used for display libraries, including phage display, cell based display (see, e.g., WO 03/029456), protein-nucleic acid fusions (see, e.g., U.S. Pat. No. 6,207,446), ribosome display (See, e.g., Mattheakis et al. (1994) Proc. Natl. Acad. Sci. USA 91:9022 and Hanes et al. (2000) Nat. Biotechnol. 18:1287-92; Hanes et al. (2000) Methods Enzymol. 328:404-30; and Schaffitzel et al. (1999) J Immunol Methods. 231(1-2):119-35), and E. coli periplasmic display (J Immunol Methods. 2005 Nov. 22; PMID: 16337958).

Phage Display: In phage display, the antibody associated with a bacteriophage, typically by linkage to a bacteriophage coat protein, which includes a nucleic acid sequence encoding the displayed antibody. The linkage can be accomplished by any of a number of approaches, such as the use of fusion protein technology (e.g., translation of a nucleic acid encoding displayed fused to the coat protein), the use of ‘cys display’ (e.g., formation of a disulfide bond between a free cysteine present on at least one chain of the antibody and a free cysteine engineered into a phage coat protein), or the use of non-covalent linkage technologies (e.g., heterodimerization domains engineered into the antibody and a phage coat protein, see, e.g., US 2003/0104355). The linkage is made using fusion protein technology, the linkage can include a flexible peptide linker, a protease site, or an amino acid incorporated as a result of suppression of a stop codon.

Phage display is described, for example, in U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; WO 90/02809; de Haard et al. (1999) J. Biol. Chem. 274:18218-30; Hoogenboom et al (1998) Immunotechnology 4:1-20; Hoogenboom et al. (2000) Immunol Today 2:371-8 and Hoet et al. (2005) Nat. Biotechnol. 23(3)344-8.

Selection: Selection of antibodies from an antibody display library generally requires that the active agent(s) in the target vaccine (e.g., the immunogen(s), e.g., the cells or viruses in a cellular vaccine) be captured or capturable (e.g., bound to a solid support or modified to allow capture of the target vaccine on a solid support, such as by biotinylation). The means by which the active agent(s) in a target vaccine are captured or made capturable will vary, depending on the preference of the practitioner and the type of active agent(s) (e.g., cellular vs. acellular). A common capture technique for cellular targets is adsorption to magnetic particles, while active agents from acellular vaccines may be modified by biotinylation and captured using a streptavidin (or avidin) derivatized solid support or directly adsorbed to the solid support (e.g., immunotubes or polystyrene multiwell plates).

The captured or capturable target vaccine is preferably treated to reduce non-specific binding of the support and target vaccine by members of the display library (especially phage, which are prone to non-specific binding). The treatment typically utilizes incubation of the immobilized target vaccine with a blocking agent, which is often a high concentration of irrelevant protein (e.g., 2-4% serum albumin, or 2% non-fat milk) but can also be a detergent (e.g., 0.1% TWEEN® 20 in PBS).

The captured or capturable target vaccine is contacted with the antibody display library to allow binding by members of the antibody display library to the active agent(s) of the target vaccine. The binding step is typically carried out under relatively non-stringent conditions (e.g., buffered saline such as PBS or tris-buffered saline), although more stringent conditions may be used (e.g., increased salt concentration or non-neutral pH (e.g., higher than about pH 8.0 or less than about pH 6.0)). The contacting step can be as short as 10 or 15 minutes, and may be extended for multiple (e.g., 2, 3, 4, or more) hours if desired.

Display library member/target vaccine complexes are then separated from unbound display library members with a washing step. If the target vaccine is capturable, then it is normally captured prior to washing. The captured target vaccine/display library members are then separated from the unbound display library members in solution and washed (e.g., by removing the solution containing unbound display library members and replacing the solution with awash solution, such as PBS or PBS with 0.1% TWEEN® 20). The wash step may be repeated (e.g., 2, 3, 4, or 5 times).

The bound display library members are then isolated. Isolation of bound display library members may be carried out by any of a variety of techniques, depending in part on the particular properties of the display library. The bound library members may be eluted from the captured target vaccine by the use of an elution buffer (e.g., 0.1 M triethylamine (TEA) or 0.1 M glycine). For libraries employing cys-display or a proteolytic cleavage site between the displayed antibody and the display means (e.g., coat protein for phage, or cell surface/cell wall protein for cell-based libraries), the appropriate reagent (e.g., a disulphide reagent for cys-display libraries or the appropriate protease for libraries having a cleavage site) can release the bound library members. When the display library is a phage library, bound library members may be recovered by infecting host bacteria with the bound library members (see, e.g., U.S. Patent Publication No. 2004/0180327).

Another method of isolation of the selected display library members is the use of competitive elution. Competitive elution utilizes a reagent (the “competitive reagent”) which contains molecules (e.g., antibodies) which bind to the target vaccine. In a preferred embodiment, the reagent utilized for competitive elution is immune serum from one or more individuals who have been exposed to the molecule/microorganism/infectious agent targeted by the target vaccine or who have been exposed to (e.g., previously immunized with) the target vaccine. In competitive elution, following the wash step(s), bound display library members are displaced or competed (eluted) off of the captured target vaccine by incubation of the display library member/captured target vaccine complexes with the competitive reagent.

The eluted or otherwise recovered display library members which bound the target vaccine are then processed to identify and characterize the displayed antibody. When the antibody display library is a phage display library, phage which have been identified/selected from a display library can be grown and harvested using standard phage preparatory methods, e.g., PEG precipitation from growth media. Display libraries in other formats can be grown and harvested in accordance with the properties of the library (e.g., propagation of the selected cells in a cell-based display library). After selection of individual display phages, the nucleic acid encoding the selected protein components can be isolated from cells infected with the selected phages or from the phage themselves, after amplification. Individual colonies or plaques can be picked, the nucleic acid isolated and sequenced. For cell-based display libraries, the vectors encoding the displayed protein can be isolated from clonal populations of cells, followed by DNA sequence analysis.

The selection process may be repeated as desired. Generally, the selection process is repeated at least once, and more typically at least twice. Selection rounds subsequent to the first round are typically performed under different conditions than the initial selection round (see, e.g., the discussion of iterative selection herein), and may even utilize a different target (e.g., to remove antibodies which react with a molecule, microorganism, or infectious agent that is different from the molecule, microorganism, or infectious agent in the target vaccine. Additionally, negative selection steps may be incorporated as well (e.g., to deplete the library of antibodies which bind to a carrier protein incorporated in the target vaccine or to deplete the library of antibodies which bind to moieties or reagents used to capture the active agent(s) of the target vaccine, such as biotin and/or streptavidin).

Display library technology may be used in an iterative selection mode. In iterative selection, antibodies which are selected from a first display library on the basis of binding the target vaccine are then varied using a mutagenesis method to form a second display library. Higher affinity binding proteins are then selected from the second library, e.g., by using higher stringency or more competitive binding and washing conditions.

In some implementations, the mutagenesis is targeted to regions at the binding interface (e.g., the CDR regions of the heavy or light chains of the identified antibodies). Further, mutagenesis can be directed to framework regions near or adjacent to the CDRs. Mutagenesis can also be limited to one or a few of the CDRs, e.g., to make precise step-wise improvements. Exemplary mutagenesis techniques include: error-prone PCR, recombination, DNA shuffling, site-directed mutagenesis and cassette mutagenesis.

In one example of iterative selection, an antibody which binds to a target vaccine with at least a minimal binding specificity for a target or a minimal activity (e.g., an equilibrium dissociation constant for binding of less than 1 nM, 10 nM, or 100 nM) is first identified from a display library. The nucleic acid sequence encoding the initial identified antibody is used as template nucleic acid for the introduction of variations, e.g., to identify one or more second generation antibodies that have enhanced properties (e.g., binding affinity, kinetics, or stability) relative to the initial antibody.

Off-Rate Selection. Since a slow dissociation rate can be predictive of high affinity, particularly with respect to interactions between polypeptides and their targets, off-rate selection can be used to select antibodies with a desired (e.g., reduced) kinetic dissociation rate for a binding interaction to a target vaccine.

To select for slow dissociating antibodies from a display library, the library is contacted to an immobilized vaccine. The immobilized vaccine is then washed with a first solution that removes non-specifically or weakly bound library members. Elution is carried out using a second solution that includes a saturating amount a free competing agent (e.g., free (non-immobilized) target vaccine, although in some cases antibodies specific to the target vaccine may be used). The free competing agent binds to library members that dissociate from the target. Rebinding is effectively prevented by the saturating amount of free competing agent to the much lower concentration of immobilized vaccine.

The second solution can have solution conditions that are substantially physiological or that are stringent. Typically, the solution conditions of the second solution are identical to the solution conditions of the first solution. Fractions of the second solution are collected in temporal order to distinguish early from late fractions. Later fractions include antibodies that dissociate at a slower rate from the target vaccine than antibodies in the early fractions.

Further, it is also possible to recover display library members that remain bound to the target vaccine even after extended incubation. These can either be dissociated using chaotropic conditions or can be amplified while attached to the target (see, e.g., U.S. Patent Publication No. 2004/0180327).

Selecting or Screening for Specificity. The display library screening methods described herein can include a selection or screening process that discards display library members that bind to a non-target entity (e.g., a cellular, viral or proteinaceous entity closely related to a cellular, viral or proteinaceous agent in the target vaccine). In one implementation, a so-called “negative selection” step is used to discriminate between the target and related non-target molecule and a related, but distinct non-target molecules. The display library or a pool thereof is contacted to the non-target entity. Members of the library (or pool thereof) that do not bind the non-target entity are collected and used in subsequent selections for binding to the target vaccine or even for subsequent negative selections. The negative selection step can be prior to or after selecting library members that bind to the target molecule.

In another implementation, a screening step is used. After display library members are isolated for binding to the target vaccine, each isolated library member is tested for its ability to bind to a non-target entity. For example, a high-throughput ELISA screen can be used to obtain this data. The ELISA screen can also be used to obtain quantitative data for binding of each isolated library member to the target vaccine as well as for cross-reactivity to entities related to the target vaccine (e.g., different strains of the organism in the target vaccine) or under different conditions (e.g., varying pH). The non-target and target binding data are compared (e.g., using a computer and software) to identify library members that have the desired binding characteristics.

Secondary Screening Methods

After selecting candidate library members that bind to a target, each candidate library member can be further analyzed, e.g., to further characterize its binding properties for the target vaccine, or for binding (or not binding) to another vaccine. Each candidate library member can be subjected to one or more secondary screening assays. The assay can be for a binding property, a catalytic property, an inhibitory property, a physiological property (e.g., cytotoxicity, renal clearance, immunogenicity), a structural property (e.g., stability, conformation, oligomerization state) or another functional property. The same assay can be used repeatedly, but with varying conditions, e.g., to determine pH, ionic, or thermal sensitivities.

As appropriate, the assays can use a display library member directly, a recombinant polypeptide produced from the nucleic acid encoding the selected polypeptide, or a synthetic peptide synthesized based on the sequence of the selected polypeptide. In the case of selected Fabs, the Fabs can be evaluated or can be modified and produced as intact IgG proteins. Exemplary assays for binding properties include enzyme-linked immunosorbent assays (ELISA), homogeneous binding assays (e.g., a solution binding assay utilizing fluorescent resonance energy transfer (FRET) as the detection system or ALPHASCREEN™ (Packard Bioscience, Meriden Conn.), which utilizes singlet oxygen generation to activate a detectable label), surface plasmon resonance (SPR) assays (see, e.g., U.S. Pat. No. 5,641,640; SPR assays are useful for measuring the equilibrium dissociation constant (K_{d}), and kinetic parameters, including Kₒₙ and K_{off}), and cellular binding assays (e.g., FACS analysis of cells expressing a target vaccine component).

Modification of Selected Antibodies

Reduction of immunogenicity of antibodies selected according the methods of the invention may be desirable, particularly when it is envisioned or expected that the passive immunotherapy will be administered to a given subject on more than one occasion, and particularly when multiple administrations are a possibility. Techniques useful for reducing immunogenicity of selected antibodies include deletion/modification of potential human T cell epitopes and ‘germlining’ of sequences outside of the CDRs (e.g., framework and Fc). Deletion of potential T cell epitopes (e.g., peptides which are predicted to bind to class II MHC) is described, for example, in International Patent Publications Nos. WO 98/52976 and WO 00/34317, and involves modifying the sequence(s) of potential T cell epitopes, commonly to an amino acid found in human germline antibody sequences at the analogous position, where such modification does not significantly alter the function of the antibody.

When a potential T cell epitope includes residues which are known or predicted to be important for antibody function (e.g., in a CDR or in a portion of framework sequence thought to be critical for antibody binding), it is preferred that changes are made outside of the important sequence, but when that is not possible, an alteration within the important sequence (e.g., in the CDR) should be made and the antibody, with and without this substitution, should be tested for function (e.g., retention of binding to the target vaccine).

Antibodies are “germlined” by reverting one or more non-germline amino acids in framework regions to corresponding germline amino acids of the antibody, so long as binding properties are substantially retained. Similar methods can also be used in the constant region, e.g., in constant immunoglobulin domains. As with removal of potential T cell epitopes, it is usually helpful to test the antibody, with and without the germline substitution(s) to be sure that the substitutions do not significantly affect antibody function. In one embodiment, as many germline residues are introduced into an isolated antibody as possible.

The germline sequence(s) used as the comparator sequence for identifying positions to be changed are commonly selected on the basis of sequence identity or similarity to antibody to be germlined. A single sequence, or a consensus sequence built from a number of germline sequences, can be used. In some cases, only a portion of the selected antibody will be germlined (e.g., CDR1 and/or CDR2 of VH).

Germline sequences of human immunoglobin genes have been determined and are available from a number of sources, including the Tomlinson et al., 1992, J. Mol. Biol. 227:776-798, Cook et al., 1995, Immunol. Today Vol. 16 (5): 237-242, Chothia et al., 1992, J. Mol. Bio. 227:799-817, the international ImMunoGeneTics information system® (IMGT), available via the world wide web at imgt.cines.fr, and the V BASE directory (compiled by Tomlinson, I. A. et al. MRC Centre for Protein Engineering, Cambridge, UK, available via the world wide web at vbase.mrc-cpe.cam.ac.uk).

Human germline sequences are disclosed in. The V BASE directory provides a comprehensive directory of human immunoglobulin variable region sequences (compiled by Tomlinson, I. A. et al. MRC Centre for Protein Engineering, Cambridge, UK).

Exemplary germline reference sequences for Vₖₐₚₚₐ include: O12/O2, O18/O8, A20, A30, L14, L1, L15, L4/18a, L5/L19, L8, L23, L9, L24, L11, L12, O11/O1, A17, A1, A18, A2, A19/A3, A23, A27, A11, L2/L16, L6, L20, L25, B3, B2, A26/A10, and A14. See, e.g., Tomlinson et al., 1995, EMBO J. 14(18):4628-3.

A germline reference sequence for the HC variable domain can be based on a sequence that has particular canonical structures, e.g., 1-3 structures in the H1 and H2 hypervariable loops. The canonical structures of hypervariable loops of an immunoglobulin variable domain can be inferred from its sequence, as described in Chothia et al., 1992, J. Mol. Biol. 227:799-817; Tomlinson et al., 1992, J. Mol. Biol. 227:776-798); and Tomlinson et al., 1995, EMBO J. 14(18):4628-38. Exemplary sequences with a 1-3 structure include: DP-1, DP-8, DP-12, DP-2, DP-25, DP-15, DP-7, DP-4, DP-31, DP-32, DP-33, DP-35, DP-40, 7-2, hv3005, hv3005f3, DP-46, DP-47, DP-58, DP-49, DP-50, DP-51, DP-53, and DP-54.

Protein Production

Standard recombinant nucleic acid methods can be used to express an antibody that is selected according to the methods of the invention. Generally, a nucleic acid sequence encoding the protein is cloned into a nucleic acid expression vector. Of course, if the protein includes multiple polypeptide chains (e.g., an IgG), each chain can be cloned into an expression vector, e.g., the same or different vectors, that are expressed in the same or different cells.

Some antibodies, e.g., Fabs, can be produced in bacterial cells, e.g., E. coli cells. The nucleic acid sequences encoding the Fab can be recloned from the display library vector into a vector adapted for expression of soluble Fabs, or, if the display vector includes a suppressible stop codon between the display entity and a bacteriophage protein (or fragment thereof), the vector nucleic acid can be transferred into a bacterial cell that cannot suppress a stop codon.

Antibodies can also be produced in eukaryotic cells. In one embodiment, the antibodies (e.g., scFv's) are expressed in a yeast cell such as Pichia (see, e.g., Powers et al., 2001, J. Immunol. Methods. 251:123-35), Hanseula, or Saccharomyces.

In one preferred embodiment, antibodies are produced in mammalian cells. Preferred mammalian host cells for expressing the clone antibodies or antigen-binding fragments thereof include Chinese Hamster Ovary (CHO cells) (including dhfr− CHO cells, described in Urlaub and Chasin, 1980, Proc. Natl. Acad. Sci. USA 77:4216-4220, used with a DHFR selectable marker, e.g., as described in Kaufman and Sharp, 1982, Mol. Biol. 159:601 621), lymphocytic cell lines, e.g., NS0 myeloma cells and SP2 cells, COS cells, HEK293T cells (J. Immunol. Methods (2004) 289(1-2):65-80), and a cell from a transgenic animal, e.g., a transgenic mammal. For example, the cell is a mammary epithelial cell.

In addition to the nucleic acid sequence encoding the diversified immunoglobulin domain, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr⁻ host cells with methotrexate selection/amplification) and the neo gene (for G418 selection).

In an exemplary system for recombinant expression of an antibody, or antigen-binding portion thereof, a recombinant expression vector encoding both the antibody heavy chain and the antibody light chain is introduced into dhfr⁻ CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium. For example, some antibodies can be isolated by affinity chromatography with a Protein A or Protein G coupled matrix.

For antibodies that include an Fc domain, the antibody production system may produce antibodies in which the Fc region is glycosylated. For example, the Fc domain of IgG molecules is glycosylated at asparagine 297 in the CH2 domain. This asparagine is the site for modification with biantennary-type oligosaccharides. It has been demonstrated that this glycosylation is required for effector functions mediated by Fcg receptors and complement Clq (Burton and Woof, 1992, Adv. Immunol. 51:1-84; Jefferis et al., 1998, Immunol. Rev. 163:59-76). In one embodiment, the Fc domain is produced in a mammalian expression system that appropriately glycosylates the residue corresponding to asparagine 297. The Fc domain can also include other eukaryotic post-translational modifications.

Antibodies can also be produced by a transgenic animal. For example, U.S. Pat. No. 5,849,992 describes a method of expressing an antibody in the mammary gland of a transgenic mammal, using a transgene including a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly.

Pharmaceutical Compositions

Antibodies selected for use in passive immunotherapeutics may be provided as compositions, e.g., pharmaceutically acceptable compositions or pharmaceutical compositions, which include an antibody selected or otherwise identified in accordance with the methods disclosed herein. The antibodies selected for use as passive immunotherapy can be formulated together with a pharmaceutically acceptable carrier.

A pharmaceutically acceptable carrier includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal, or epidermal administration (e.g., by injection or infusion), although carriers suitable for inhalation and intranasal administration are also contemplated.

Antibodies selected for use in passive immunotherapeutics may be formulated as a acceptable salt, which is a salt that retains the desired biological activity of the parent compound and does not impart any undesired toxicological effects (see e.g., Berge, S. M., et al., 1977, J. Pharm. Sci. 66:1-19). Examples of such salts include acid addition salts (e.g., salts derived from nontoxic inorganic acids, such as hydrochloric or phosphoric, or from nontoxic organic acids, such as aliphatic mono- and dicarboxylic acids), base addition salts (e.g., salts derived from alkaline earth metals, such as sodium, potassium, magnesium, calcium; or from nontoxic organic amines, such as N,N′-dibenzylethylenediamine, ethylenediamine, and the like).

The compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The form can depend on the intended mode of administration and therapeutic application. Many compositions are in the form of injectable or infusible solutions, such as compositions similar to those used for administration of humans with antibodies. An exemplary mode of administration is parenteral (e.g., intravenous, subcutaneous, intraperitoneal, intramuscular). Exemplary routes of administration for passive immunotherapeutics containing antibodies selected by the methods disclosed herein include intravenous infusion or injection, and intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

The composition can be formulated as a solution, microemulsion, dispersion, liposome, or other ordered structure suitable to high drug concentration. Sterile injectable solutions can be prepared by incorporating the antibody in the required amount in an appropriate solvent with one or a combination of ingredients enumerated above, as required, followed by filtered sterilization. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

Passive immunotherapeutics can be administered by a variety of methods, although for many applications, the preferred route/mode of administration is parenteral injection or infusion (e.g., intravenous, intramuscular, or subcutaneous). For example, passive immunotherapeutics can be administered by intravenous infusion at a rate of less than 30, 20, 10, 5, or 1 mg/min to reach a dose of about 1 to 100 mg/m² or 7 to 25 mg/m² of the antibody, The route and/or mode of administration will vary depending upon the desired results. In certain embodiments, the antibody may be formulated in a controlled release formulation, such as a microencapsulated delivery system. Biodegradable, biocompatible polymers can be used, such as ethylene vinyl acetate, polyanhydrides, polyglycolic acid, collagen, polyorthoesters, and polylactic acid (see, e.g., Sustained and Controlled Release Drug Delivery Systems, J. R. Robinson, ed., 1978, Marcel Dekker, Inc., New York.)

Pharmaceutical compositions can be administered with medical devices. For example, in one embodiment, a pharmaceutical composition disclosed herein can be administered with a device, e.g., a needleless hypodermic injection device, a pump, or implant.

Dosage regimens are adjusted to provide the desired response (e.g., a therapeutic response). For example, a single bolus may be administered, several divided doses may be administered over time or the dose may be proportionally reduced or increased as indicated by the exigencies of the therapeutic situation.

An exemplary, non-limiting range for a therapeutically or prophylactically effective amount of a passive immunotherapeutic containing an antibody selected/identified in accordance with the methods disclosed herein is 0.1-20 mg/kg, more preferably 1-10 mg/kg. Dosage values may vary with the type and severity of the condition to be alleviated. For a particular subject, specific dosage regimens can be adjusted over time according to the individual need and the professional judgment of the person administering or supervising the administration of the compositions.

The pharmaceutical compositions disclosed herein may include a “therapeutically effective amount” or a “prophylactically effective amount” of an antibody selected/identified in accordance with the methods disclosed herein. A “therapeutically effective amount” refers to an amount effective, at dosages and for periods of time necessary, to achieve the desired therapeutic result. A therapeutically effective amount of the composition may vary according to factors such as the disease state, age, sex, and weight of the individual, and the ability of the protein to elicit a desired response in the individual. A therapeutically effective amount is also one in which any toxic or detrimental effects of the composition is outweighed by the therapeutically beneficial effects.

A “prophylactically effective amount” refers to an amount effective, at dosages and for periods of time necessary, to achieve the desired prophylactic result. Typically, because a prophylactic dose is used in subjects prior to or at an earlier stage of disease, the prophylactically effective amount will be less than the therapeutically effective amount.

Stabilization and Retention

In one embodiment, an antibody for use in a passive immunotherapeutic is physically associated with a moiety that improves its stabilization and/or retention in circulation, e.g., in blood, serum, lymph, or other tissues, e.g., by at least 1.5, 2, 5, 10, or 50 fold. For example, an antibody for use in a passive immunotherapeutic can be associated with a polymer, e.g., a substantially non-antigenic polymers, such as polyalkylene oxides or polyethylene oxides. Suitable polymers will vary substantially by weight. Polymers having molecular number average weights ranging from about 200 to about 35,000 (or about 1,000 to about 15,000, and 2,000 to about 12,500) can be used. For example, an antibody for use in a passive immunotherapeutic can be conjugated to a water soluble polymer, e.g., hydrophilic polyvinyl polymers, e.g. polyvinylalcohol and polyvinylpyrrolidone. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. An antibody for use in a passive immunotherapeutic can also be associated (e.g., produced as a fusion protein) with a carrier protein, e.g., a serum albumin, such as a human serum albumin.

Kits

A passive immunotherapeutic containing an antibody selected/identified in accordance with the methods disclosed herein can be provided in a kit, e.g., as a component of a kit. For example, the kit includes (a) an antibody selected in accordance with the methods disclosed herein, and, optionally (b) informational material. The informational material can be descriptive, instructional, marketing or other material that relates to the antibody or the use of the antibody as a passive immunotherapy.

Treatments

Passive immunotherapeutic compositions incorporating antibodies identified by the methods disclosed herein are useful in the treatment and prevention of disease. While vaccines are generally effective at preventing disease, most vaccines have a marginal effect on a disease or disorder, particularly infectious diseases, once the disease or disorder has manifested clinically. Additionally, vaccines are largely ineffective and can be dangerous in immunocompromised (e.g., immunosuppressed or immunodeficient) individuals.

A passive immunotherapeutic incorporating antibodies identified by the methods disclosed herein can be administered to subjects for prophylaxis or treatment of the disease or disorder targeted by the target vaccine used to identify the antibodies in the passive immunotherapeutic. Accordingly, passive immunotherapeutics incorporating antibodies identified by the methods disclosed herein are useful for the prophylaxis and/or treatment of disorders including, but not limited to, anthrax (Bacillus anthracis infection), hepatitis A, hepatitis B, polio, smallpox, bacterial meningitis (Streptococcus pneumoniae, Neisseria meningitidis, or Haemophilus influenzae infection), diptheria (Corynebacterium diphtheriae infection), influenza, measles, mumps, pertussis (Bordetella pertussis infection), pneumococcal pneumonia (Streptococcus pneumoniae infection), ricin toxin intoxication, botulisim, tetanus (Clostricium tetani infection), rotaviral diarrhea (rotavirus infection), rubella, typhus, rabies, human papillomavirus (HPV) infection, cervical cancer associated with HPV infection, chickenpox, and shingles.

Methods of administering passive immunotherapeutics are also described in “Pharmaceutical Compositions.” Suitable dosages of the molecules used can depend on the age and weight of the subject and the particular drug used. Depending on the particular disease or disorder targeted by the passive immunotherapy, the dose may be about 0.1, 1.0, 3.0, 6.0, or 10.0 mg/Kg. For an IgG having a molecular mass of 150,000 g/mole (two binding sites), these doses correspond to approximately 18 nM, 180 nM, 540 nM, 1.08 μM, and 1.8 μM of binding sites for a 5 L blood volume.

The contents of all references, pending patent applications and published patents, cited throughout this application are hereby expressly incorporated by reference.

A number of embodiments of the invention have been described. Nevertheless, it will be understood that various modifications may be made without departing from the spirit and scope of the invention. Accordingly, other embodiments are within the scope of the following claims.

What is claimed is:

1. A method for identifying one or more antibodies for use in an passive immunotherapy, comprising
selecting antibodies from an antibody display library which bind to a target vaccine by contacting the target vaccine with members of the antibody display library and,
identifying the members of the antibody display library which bind the target vaccine, to thereby identify one or more antibodies for use in a passive immunotherapy.

2. The method of claim 1, wherein the target vaccine is a cellular vaccine or an acellular vaccine.

3. The method of claim 2, wherein the acellular vaccine is selected from a cell-free cellular extracts, a cell-free purified or fractionated cellular component, and a conjugate of a purified or fractionated cellular component to a carrier moiety.

4. The method of claim 2, wherein the vaccine is cellular vaccine and the cellular vaccine is a live vaccine.

5. The method of claim 4, wherein the live vaccine is selected from an anthrax vaccine, a smallpox vaccine and a rotavirus vaccines.

6. The method of claim 2, wherein the vaccine is cellular vaccine and the cellular vaccine is an attenuated vaccine.

7. The method of claim 6, wherein the attenuated vaccine is selected from a Sabin oral polio vaccine (OPV), an attenuated influenza A/B vaccine, a measles vaccine, a mumps vaccine, a rubella vaccine and a chickenpox/shingles vaccine.

8. The method of claim 2, wherein the cellular vaccine is a killed vaccine.

9. The method of claim 8, wherein the killed vaccine is selected from an inactivated hepatitis A whole virus vaccine, a Salk polio vaccine, an injected influenza A/B vaccine, and a killed rabies virus vaccine.

10. The method of claim 2, wherein the target vaccine is an acellular vaccine and the acellular vaccine is selected from a toxoid, a cell wall component, and a proteinaceous component of the microorganism or infectious agent targeted by the vaccine.

11. The method of claim 10, wherein the acellular vaccine is adsorbed or is conjugated to a carrier moiety.

12. The method of claim 2, wherein the target vaccine is an acellular vaccine and the acellular vaccine is selected from an acellular anthrax vaccine, a hepatitis B surface antigen vaccine, a diptheria toxoid vaccine, a Haemophilus influenzae type B capsular polysaccharide conjugate vaccine, a Streptococcus pneumoniae capsular polysaccharide vaccine, a capsular polysaccharide/diptheria toxin CRM₁₉₇ conjugate vaccine, a Neisseria meningitidis multivalent capsular extract vaccine, a pertussis vaccine, a tetanus vaccine, a typhus vaccine, a Lyme's disease vaccine, a human papillomavirus (HPV) vaccine, a ricin toxin or toxoid and Clostridium botulinum toxin or toxoid.

13. The method of claim 1, wherein the step of identifying members of the antibody display library which bind the target vaccine includes eluting bound display library members from the target vaccine by bulk elution which elutes essentially all of the antibody display library members bound to the target vaccine.

14. The method of claim 1, wherein the step of identifying the members of the antibody display library which bind the target vaccine includes eluting the bound display library members from the target vaccine by competition, using a competition reagent containing molecules which bind to the target vaccine.

15. The method of claim 14, wherein the competition reagent is an antibody to an active agent in the target vaccine is immune serum derived from one or more individuals who have been exposed to the molecule, microorganism, or infectious agent targeted by the target vaccine.

16. The method of claim 1, wherein the step of identifying members of the antibody display library which bind the target vaccine also includes obtaining the nucleotide sequence(s) of the nucleic acid(s) which encodes the antibody displayed on the surface of antibody display library members being identified.

17. The method of claim 1, further comprising determining the affinity or specificity of the identified members.

18. The method of claim 1, wherein the target vaccine is a univalent or multivalent.

19. A passive immunotherapeutic composition comprising one or more antibodies identified by a method comprising:
selecting antibodies from an antibody display library which bind to a target vaccine by contacting the target vaccine with members of the antibody display library and,
identifying the members of the antibody display library which bind the target vaccine, to thereby identify one or more antibodies for use in a passive immunotherapy.

20. A kit for passive immunotherapy comprising one or more antibodies identified by a method comprising:
selecting antibodies from an antibody display library which bind to a target vaccine by contacting the target vaccine with members of the antibody display library and,
identifying the members of the antibody display library which bind the target vaccine, to thereby identify one or more antibodies for use in a passive immunotherapy.

21. A method of prophylaxis or treatment of a disorder targeted by a target vaccine, comprising
administering to a subject at risk of, exposed to, or having the disease or disorder targeted by the target vaccine a passive immunotherapy composition comprising one or more antibodies identified by a method that comprises:
selecting antibodies from an antibody display library which bind to a target vaccine by contacting the target vaccine with members of the antibody display library and,
identifying the members of the antibody display library which bind the target vaccine, to identify one or more antibodies for use in a passive immunotherapy composition,
thereby preventing or treating the disorder.
